(19) United States
(12) Patent Application Publication
(10) Pub. No.: US 2009/0136578 A1
(43) Pub. Date: May 28, 2009

(54) Pharmaceutical Composition Comprising Perindopril or Its Salts

(75) Inventor: Peter Svete, Borovnica (SI)

Correspondence Address: ARENT FOX LLP, 1050 CONNECTICUT AVENUE, N.W., SUITE 400, WASHINGTON, DC, 20036, US

(73) Assignee: LEK Pharmaceutical d.d., Ljublijana (SI)

(21) Appl. No.: 11/991,201

(22) PCT Filed: Aug. 28, 2006
(86) PCT No.: PCT/EP2006/008400

§ 371 Date: Feb. 28, 2008

(30) Foreign Application Priority Data
Aug. 30, 2005 (SI) P200500244

(51) Int. Cl. A61K 9/14; A61K 31/403

(52) U.S. Cl. 424/489; 514/412

(57) ABSTRACT

The present invention relates to a stable pharmaceutical composition of the ACE inhibitor perindopril or its salts having a defined particle size distribution.

The present invention relates to a stable pharmaceutical composition of the ACE inhibitor perindopril or its salts.

Perindopril and its pharmaceutically acceptable salts are known as angiotensin converting enzyme inhibitors and are used in the treatment of cardiovascular diseases, especially in the treatment of hypertension and heart failure. Perindopril is chemically known as (2S,3aS,7aS)-((2-(1-(ethoxycarbonyl)-(S)-butylamino)-(S)-propionyl)octahydro-indole-2-carboxylic acid and can be represented by formula (I).

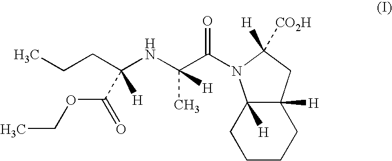

Perindopril is known, for example, from EP-A 0049658; the tert-butylamine salt thereof, i.e. perindopril erbumine, is known from EP-A 0308 341.

It is known that ACE inhibitors are susceptible to degradation via a) hydrolysis of the side-chain ester group, b) intramolecular cyclization to form diketopiperazines, c) isomerisation at some chiral centres and d) oxidation to form colored products. Perindopril is especially susceptible to hydrolysis and to intramolecular cyclization due to its molecular structure.

The main degradation products of perindopril are diketopiperazine (ethyl(2S)-2-[(3S,5aS,9aS,10aS)-3-methyl-1,4-dioxodecahydropyrazino[1,2-a]indol-2(1H)-yl]pentanoate), known as impurity F in European Pharmacopea 5.0, obtained after intramolecular cyclization, and perindoprilate ((2S,3aS,7aS)-1-[(2S)-2-[[(1S)-1-carboxybutyl]amino]propanoyl]octahydro-1H-indole-2-carboxylic acid), known as impurity B in European Pharmacopea 5.0, obtained after hydrolysis of side-chain ester group.

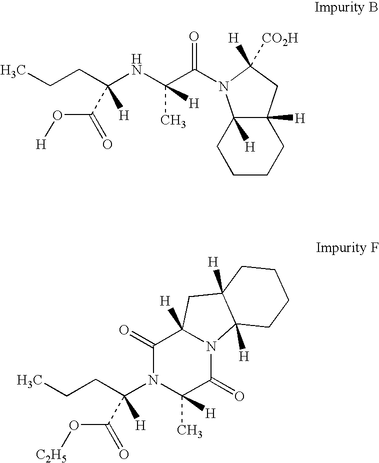

Different methods of stabilizing ACE inhibitors in pharmaceutical compositions are known in the prior art. For example, pharmaceutical compositions comprising ACE inhibitors can be stabilized by the presence of alkali or alkaline metal salts (WO 01/15724, EP 280 999), magnesium oxide (WO 99/62560), hydrochloric acid donors (EP 468 929), ascorbic acid (EP 264 888).

Furthermore, effects of different pharmaceutical excipients on the stability of ACE inhibitors have been also disclosed in the prior art.

EP 408 273 discloses that stability of fosinopril sodium tablets is increased by use of sodium stearyl fumarate or hydrogenated vegetable oil as lubricant instead of magnesium stearate.

U.S. Pat. No. 5,562,921 discloses that enalapril maleate is particularly unstable in the presence of some usual pharmaceutical excipients such as microcrystalline cellulose, starch and calcium phosphate, and also in the presence of magnesium stearate. Little decomposition is observed by use of water-soluble carbohydrate excipients, such as lactose, compressible sugars, dextrates, dextrose, dextrin, mannitol and sorbitol and by use of zinc stearate or glyceryl monostearate as lubricant.

WO 03/028707 discloses that use of lactose monohydrate as diluent enables better stability of solid oral pharmaceutical compositions of ramipril than use of anhydrous lactose or starch.

GB 2 394 660 discloses that the presence of colloidal silicon dioxide promotes the degradation of ACE inhibitors in pharmaceutical compositions.

However, the problem of the stability of pharmaceutical composition comprising ACE inhibitors has not been solved completely. Therefore, there is still a need to develop a stable pharmaceutical composition comprising ACE inhibitors, particularly perindopril erbumine.

The Applicant has found that particle size of perindopril erbumine is crucial factor having a high impact on the stability of its pharmaceutical composition. Particularly, it was found that the stability of pharmaceutical composition of perindopril erbumine having large particles is higher in comparison to the stability of pharmaceutical composition of perindopril erbumine having small particles. Thus, the present invention provides a stable pharmaceutical composition of perindopril erbumine having a defined particle size distribution.

As used herein, the term “small particles”, when used in reference to the size of perindopril erbumine particles, indicates a particle size with median particle diameter lower than 5 μm, preferably the term “small particles” indicates a particle size distribution in which 10% or fewer of the particles have a diameter below about 0.8 μm, 10% or fewer of the particles have a diameter above about 6 μm, and the median particle diameter is from about 2 to about 3 μm.

As used herein, the term “large particles”, when used in reference to the size of perindopril erbumine particles, indicates a particle size with median particle diameter above 7 μm, preferably the term “large particles” indicates a particle size with median particle diameter from 8 μm to 50 μm, more preferably the term “large particles” indicates a particle size with median particle diameter from 8 μm to 20 μm, most preferably the term “large particles” indicates a particle size distribution in which 10% or fewer of the particles have a diameter below about 2 μm, 10% or fewer of the particles have a diameter above about 30 μm, and the median particle diameter is from about 10 to about 15 μm.

As used herein, the term “median”, when used in reference to the size of perindopril erbumine particles, indicates that about 50% of all measurable particles measured have a particle size less than the defined median particle size value, and about 50% of all measurable particles measured have a particle size greater than the defined median particle size value.

In accordance with the invention, the size distribution of perindopril erbumine particles is determined by laser diffraction. The method of determining the size of perindopril erbumine particles used a Malvern™ Mastersizer S laser diffraction instrument. 100 mg of perindopril erbumine sample were suspended in 10 ml of hexane. The suspensions were mixed and then sonicated for 60 seconds to thoroughly disperse the perindopril erbumine particles. The dispersion was then circulated in the flow cell of the Malvern™ Mastersizer for two minutes before particle size measurements were taken.

A pharmaceutical composition in the form of a tablet or a capsule may comprise in addition to active pharmaceutical ingredient one or more pharmaceutically acceptable excipients (inactive ingredients), such as fillers, disintegrants, glidants, lubricants, etc.

During the development of the stable pharmaceutical composition of perindopril erbumine comparative tests were performed to investigate effects of various pharmaceutically acceptable excipients e.g. fillers, disintegrants and glidants, and different particle size of perindopril erbumine on the stability of perindopril erbumine.

For the comparative tests, samples of perindopril erbumine with different particle size were mixed with some common pharmaceutically acceptable excipients in binary or ternary mixtures. Pharmaceutical compositions, particularly tablets, with perindopril erbumine having different particle size were prepared as well. Binary and ternary mixtures and tablets were exposed to the stress condition, e.g. 60° C. for 14 days or 40° C./75% relative humidity for 1 month. Degradation products of perindopril were determined using HPLC method as described in European Pharmacopea 5.0 (January 2005, monograph for Perindopril tert-butylamine—pages 2210-2212).

Through comparative tests using various combinations of perindopril erbumine having different particle size and pharmaceutically acceptable excipients, it was surprisingly found that the degradation of perindopril erbumine having large particles is smaller in comparison to the degradation of perindopril erbumine having small particles regardless of the used pharmaceutically acceptable excipients or testing conditions employed.

Moreover, it was found that there is no significant difference in dissolution profile of tablets comprising large particles of perindopril erbumine in comparison to tablets comprising small particles of perindopril erbumine. Consequently, a particle size should not have an effect on a bioavailability of perindopril erbumine.

Therefore, the first embodiment of the present invention is related to a stable pharmaceutical composition comprising perindopril erbumine having particle size with median particle diameter above 7 μm.

In another embodiment the present invention relates to a stable pharmaceutical composition comprising perindopril erbumine having particle size with median particle diameter from 8 μm to 50 μm.

In another embodiment the present invention relates to a stable pharmaceutical composition comprising perindopril erbumine having particle size with median particle diameter from 8 μm to 20 μm.

In another embodiment the present invention relates to a stable pharmaceutical composition comprising perindopril erbumine having a particle size distribution in which 10% or fewer of the particles have a diameter below about 2 μm, 10% or fewer of the particles have a diameter above about 30 μm, and the median particle diameter is from about 10 to about 15 μm.

Furthermore, when testing a compatibility of various fillers with perindopril erbumine, it was surprisingly found, in contrast to the prior art (U.S. Pat. No. 5,562,921), that microcrystalline cellulose is more compatible with perindopril erbumine than lactose.

Therefore, in another embodiment the present invention relates to a pharmaceutical composition comprising perindopril erbumine having particle size with median particle diameter above 7 μm, preferably from 8 μm to 50 μm, more preferably from 8 μm to 20 μm, and microcrystalline cellulose as filler.

In another embodiment, the present invention relates to a pharmaceutical composition comprising perindopril erbumine having a particle size distribution in which 10% or fewer of the particles have a diameter below about 2 μm, 10% or fewer of the particles have a diameter above about 30 μm, and the median particle diameter is from about 10 to about 15 μm and microcrystalline cellulose as filler.

Some additional pharmaceutical excipients can be added into the pharmaceutical composition of perindopril erbumine in order to improve its technological properties like powder flowability and compressibility of the dry mixture containing active ingredient and excipients and to attain the desired release rate of perindopril erbumine from pharmaceutical composition.

Pharmaceutical composition of the present invention may contain one or more additional pharmaceutical excipients such as additional fillers, binders, disintegrants, glidants, lubricants, etc.

Suitable additional filler may be selected from the group consisting of silicified microcrystalline cellulose, e.g. Prosolv, powdered cellulose, starch, pregelatinized starch, sucrose, glucose, mannitol, sorbitol, calcium phosphate, calcium hydrogen phosphate, aluminium silicate, sodium chloride, potassium chloride, calcium carbonate, calcium sulphate, dextrates, dextrin, maltodextrin, glycerol palmitostearate, hydrogenated vegetable oil, kaolin, magnesium carbonate, magnesium oxide, polymethacrylates, talc, and others. Preferred additional filler is silicified microcrystalline cellulose.

Suitable binder may be selected from the group consisting of starch, pregelatinized starch, gelatine, sodium carboxymethylcellulose, methylcellulose, hydroxyethylcellulose, hydroxypropylcellulose, hydroxypropylmethylcellulose, ethylcellulose, polyvinylpyrrolidone, alginic acid, sodium alginate, acacia, carbomer, dextrin, guar gum, hydrogenated vegetable oil, glucose syrup, magnesium aluminium silicate, maltodextrin, polymethacrylates, zein.

Suitable disintegrant may be selected from the group consisting of starch, pregelatinized starch, sodium starch glycolate, sodium carboxymethylcellulose, cross-linked sodium carboxymethylcellulose, calcium carboxymethylcellulose, methylcellulose, powdered cellulose, silicified microcrystalline cellulose, polacrilin potassium, e.g. Amberlit, cross-linked polyvinylpyrrolidone, alginic acid, sodium alginate, colloidal silicon dioxide, guar gum, magnesium aluminium silicate, and others. Preferred disintegrants are silicified microcrystalline cellulose and polacrilin potassium.

Suitable glidant may be selected from the group consisting of magnesium stearate, calcium stearate, aluminium stearate, stearic acid, palmitic acid, cetanol, stearol, polyethylene glycols of different molecular weights, magnesium trisilicate, calcium phosphate, colloidal silicon dioxide, e.g. Aerosil, micronized silicon dioxide, e.g. Syloid, talc, powdered cellulose, starch and others. Preferred glidants are colloidal silicon dioxide and micronized silicon dioxide.

Suitable lubricant may be selected from the group consisting of stearic acid, calcium, magnesium, zinc or aluminium stearate, siliconized talc, glycerol monostearate, glycerol palmitostearate, hydrogenated castor oil, hydrogenated vegetable oil, mineral oil, light mineral oil, polyethylene glycol, sodium benzoate, sodium lauryl sulphate, sodium stearyl fumarate, talc and others. Preferred lubricant is magnesium stearate.

In another embodiment the present invention relates to a pharmaceutical composition comprising:
1-20% w/w of perindopril erbumine having particle size with median particle diameter above 7 μm, preferably from 8 μm to 50 μm, more preferably from 8 μm to 20 μm,
30-60% w/w of microcrystalline cellulose,
30-60% w/w of silicified microcrystalline cellulose,
0-5% w/w of polacrilin potassium,
0-5% w/w of colloidal silicon dioxide,
0-5% w/w of micronized silicon dioxide, and
0-5% w/w of magnesium stearate.

In another embodiment the present invention relates to a pharmaceutical composition comprising:
2-8% w/w of perindopril erbumine having particle size with median particle diameter above 7 μm, preferably from 8 μm to 50 μm, more preferably from 8 μm to 20 μm,
40-50% W/W of microcrystalline cellulose,
40-50% w/w of silicified microcrystalline cellulose,
0.5-2% W/W of polacrilin potassium,
0-1% w/w of colloidal silicon dioxide,
0.5-2% w/w of micronized silicon dioxide, and
0.5-2% w/w of magnesium stearate.

In another embodiment the present invention relates to a pharmaceutical composition comprising:
1-20% w/w of perindopril erbumine having particle size distribution in which 10% or fewer of the particles have a diameter below about 2 μm, 10% or fewer of the particles have a diameter above about 30 μm, and the median particle diameter is from about 10 to about 15 μm,
30-60% w/w of microcrystalline cellulose,
30-60% w/w of silicified microcrystalline cellulose,
0-5% w/w of polacrilin potassium,
0-5% w/w of colloidal silicon dioxide,
0-5% w/w of micronized silicon dioxide, and
0-5% w/w of magnesium stearate.

In another embodiment the present invention relates to a pharmaceutical composition comprising:
2-8% w/w of having particle size distribution in which 10% or fewer of the particles have a diameter below about 2 μm, 10% or fewer of the particles have a diameter above about 30 μm, and the median particle diameter is from about 10 to about 15 μm,
40-50% w/w of microcrystalline cellulose,
40-50% w/w of silicified microcrystalline cellulose,
0.5-2% w/w of polacrilin potassium,
0-1% w/w of colloidal silicon dioxide,
0.5-2% w/w of micronized silicon dioxide, and
0.5-2% w/w of magnesium stearate.

A pharmaceutical composition of the present invention comprises from about 1 to about 20 mg of perindopril erbumine, preferably from 2 to 8 mg of perindopril erbumine, more preferably 2, 4 or 8 mg of perindopril erbumine.

Optionally, the pharmaceutical compositions of the present invention may be combination products comprising one or more additional pharmaceutically active components in addition to perindopril erbumine. Preferably, an additional pharmaceutically active component is a diuretic, e.g. indapamide.

In another embodiment the present invention relates to use of the pharmaceutical composition of the present invention for the preparation of a medicament for use in the treatment of cardiovascular diseases, e.g. hypertension or heart failure.

In another embodiment the present invention relates to a method for the treatment of cardiovascular diseases, e.g. hypertension or heart failure, comprising administering the pharmaceutical composition of the present invention.

The following examples illustrate the invention, but do not limit it in any way:

### EXAMPLE 1

Samples of perindopril erbumine having large or small particles were mixed with microcrystalline cellulose (Avicel) into the binary mixtures having a ratio perindopril erbumine:Avicel=1:2. Binary mixtures were closed into vials and exposed to the stress condition of 60° C. for 14 days. Degradation products (impurities B and F) of perindopril were determined using HPLC method as described in European Pharmacopea 5.0 (p. 2210-2212). Results of the HPLC analysis are presented in the table below.

| | perindopril erbumine - | | perindopril erbumine - | |
|---|---|---|---|---|
| | small particles | | large particles | |
| | beginning | 60° C., 14 days | beginning | 60° C., 14 days |
| sum of | <0.05% | 0.81% | <0.05% | 0.07% |
| impurities | | | | |
| impurity B | <0.05% | <0.05% | <0.05% | <0.05% |
| impurity F | <0.05% | 0.74% | <0.05% | 0.07% |

### EXAMPLE 2

Samples of perindopril erbumine having large or small particles were mixed with anhydrous lactose into the binary mixtures having a ratio perindopril erbumine:anhydrous lactose=1:2. Binary mixtures were closed into vials and exposed to the stress condition of 60° C. for 14 days. Degradation products (impurities B and F) of perindopril were determined using HPLC method as described in European Pharmacopea 5.0 (p. 2210-2212). Results of the HPLC analysis are presented in the table below.

| | perindopril erbumine - | | perindopril erbumine - | |
|---|---|---|---|---|
| | small particles | | large particles | |
| | beginning | 60° C., 14 days | beginning | 60° C., 14 days |
| sum of | <0.05% | 0.88% | <0.05% | 0.13% |
| impurities | | | | |
| impurity B | <0.05% | <0.05% | <0.05% | <0.05% |
| impurity F | <0.05% | 0.77% | <0.05% | 0.13% |

### EXAMPLE 3

Samples of perindopril erbumine having large or small particles were mixed with colloidal anhydrous silicon dioxide (Aerosil) into the binary mixtures having a ratio perindopril erbumine:Aerosil=15:1. Binary mixtures were closed into vials and exposed to the stress condition of 60° C. for 14 days. Degradation products (impurities B and F) of perindopril were determined using HPLC method as described in European Pharmacopea 5.0 (p. 2210-2212). Results of the HPLC analysis are presented in the table below.

| | perindopril erbumine - | | perindopril erbumine - | |
|---|---|---|---|---|
| | small particles | | large particles | |
| | beginning | 60° C., 14 days | beginning | 60° C., 14 days |
| sum of | <0.05% | 0.80% | <0.05% | 0.08% |
| impurities | | | | |
| impurity B | <0.05% | <0.05% | <0.05% | <0.05% |
| impurity F | <0.05% | 0.80% | <0.05% | 0.08% |

### EXAMPLE 4

Samples of perindopril erbumine having large or small particles were mixed with microcrystalline cellulose (Avicel) and micronized silicon dioxide (Syloid) into the ternary mixtures having ratio perindopril erbumine:Avicel:Syloid=2:10:1. Ternary mixtures were exposed to the stress condition of 40° C./75% relative humidity for 1 month. Degradation products (impurities B and F) of perindopril were determined using HPLC method as described in European Pharmacopea 5.0 (p. 2210-2212). Results of the HPLC analysis are presented in the table below.

| | perindopril erbumine - | | perindopril erbumine - | |
|---|---|---|---|---|
| | small particles | | large particles | |
| | | 40° C./75% RH, | | 40° C./75% RH, |
| | beginning | 1 month | beginning | 1 month |
| sum of | 0.11% | 0.36% | 0.11% | 0.29% |
| impurities | | | | |
| impurity B | <0.05% | 0.14% | <0.05% | 0.05% |
| impurity F | <0.05% | 0.17% | <0.05% | 0.18% |

### EXAMPLE 5

Samples of perindopril erbumine having large or small particles were mixed with anhydrous lactose and micronized silicon dioxide (Syloid) into the ternary mixtures having ratio perindopril erbumine:anhydrous lactose:Syloid=2:10:1. Ternary mixtures were exposed to the stress condition of 40° C./75% relative humidity for 1 month. Degradation products (impurities B and F) of perindopril were determined using HPLC method as described in European Pharmacopea 5.0 (p. 2210-2212). Results of the HPLC analysis are presented in the table below.

| | perindopril erbumine - | | perindopril erbumine - | |
|---|---|---|---|---|
| | small particles | | large particles | |
| | | 40° C./75% RH, | | 40° C./75% RH, |
| | beginning | 1 month | beginning | 1 month |
| sum of | 0.10% | 2.75% | 0.10% | 1.92% |
| impurities | | | | |
| impurity B | <0.05% | 1.24% | <0.05% | 1.09% |
| impurity F | <0.05% | 1.35% | <0.05% | 0.70% |

### EXAMPLE 6

Samples of perindopril erbumine having large or small particles were mixed with selected pharmaceutical excipients in stated ratio:

| | |
|---|---|
| perindopril erbumine (large or small particles): | 4.5% |
| microcrystalline cellulose: | 44.5% |
| silicified microcrystalline cellulose: | 47.5% |
| polacrilin potassium: | 1.0% |
| micronized silicon dioxide: | 1.0% |
| colloidal silicon dioxide: | 0.5% |
| Mg stearate: | 1.0% |

Mixture was homogenized and pressed into the tablets. The amount of the perindopril erbumine in the tablets was defined by the mass of the tablet. Tablet could contain 2, 4 or 8 mg of perindopril erbumine.

Tablets were exposed to the stress condition of 60° C. for 14 days and 40° C./75% relative humidity for 1 month. Degradation products (impurities B and F) of perindopril were determined using HPLC method as described in European Pharmacopea 5.0 (p. 2210-2212). Results of the HPLC analysis are presented in the table below.

| | perindopril erbumine - | | | perindopril erbumine - | | |
|---|---|---|---|---|---|---|
| | small particles | | | large particles | | |
| | | 60° C., | 40° C./75% RH, | | 60° C., | 40° C./75% RH, |
| | beginning | 14 days | 1 month | beginning | 14 days | 1 month |
| sum of impurities | 0.05% | 1.79% | 0.65% | 0.07% | 1.40% | 0.41% |
| impurity B | <0.05% | 0.27% | 0.09% | <0.05% | 0.56% | 0.13% |
| impurity F | <0.05% | 0.96% | 0.51% | 0.07% | 0.63% | 0.28% |

### EXAMPLE 7

Dissolution tests of tablets from example 6 (containing perindopril erbumine having large or small particles) were conducted in 900 mL 0.1 N HCl at 37° C. using USP apparatus 2 (paddle) at 50 rpm with serial sampling at 5, 10, 15 and 45 minutes. Concentration of perindopril was determined using HPLC method as described in European Pharmacopea 5.0 (p. 2210-2212).

Results of the HPLC analysis are presented in the table below.

| | sample | |
|---|---|---|
| | tablets perindopril erbumine - | tablets perindopril erbumine - |
| time (min) | small particles | large particles |
| 5 | 90.1 | 89.8 |
| 10 | 97.2 | 98.1 |
| 15 | 99.0 | 99.3 |
| 45 | 100 | 100 |

1. A pharmaceutical composition comprising perindopril erbumine having a particle size with median particle diameter above 7 μm.

2. A pharmaceutical composition according to claim 1 comprising perindopril erbumine having a particle size with median particle diameter from 8 to 50 μm.

3. A pharmaceutical composition according to claim 1 comprising perindopril erbumine having a particle size with median particle diameter from 8 to 20 μm.

4. A pharmaceutical composition according to claim 1 comprising perindopril erbumine having a particle size distribution in which 10% or fewer of the particles have a diameter below about 2 μm, 10% or fewer of the particles have a diameter above about 30 μm, and the median particle diameter is from about 10 to about 15 μm.

5. A pharmaceutical composition comprising perindopril erbumine according to claim 1, wherein said pharmaceutical composition comprises microcrystalline cellulose as filler.

6. A pharmaceutical composition according to claim 1 comprising:
1-20% w/w of perindopril erbumine,
30-60% w/w of microcrystalline cellulose,
30-60% w/w of silicified microcrystalline cellulose,
0-5% w/w of polacrilin potassium,
0-5% w/w of colloidal silicon dioxide,
0-5% w/w of micronized silicon dioxide, and
0-5% W/w of magnesium stearate.

7. A pharmaceutical composition according to claim 1 comprising from 1 to 20 mg of perindopril erbumine.

8. A pharmaceutical composition according to claim 1 comprising one or more additional pharmaceutically active component.

9. A pharmaceutical composition according to claim 8 wherein said additional pharmaceutically active component is a diuretic.

10. A pharmaceutical composition according to claim 9 wherein said diuretic is indapamide.

11. A method of using the pharmaceutical composition according to claim 1 for the preparation of a medicament for use in the treatment of cardiovascular diseases.
